(19) United States
(12) Patent Application Publication
(10) Pub. No.: US 2020/0383842 A1
(43) Pub. Date: Dec. 10, 2020

(54) STRETCH SHEET FOR ABSORBENT ARTICLE AND METHOD FOR PRODUCING THE SAME

(71) Applicant: Kao Corporation, Tokyo (JP)

(72) Inventors: Reiko OONISHI, Utsunomiya-shi, Tochigi (JP); Yusuke URAYAMA, Utsunomiya-shi, Tochigi (JP)

(73) Assignee: Kao Corporation, Tokyo (JP)

(21) Appl. No.: 16/613,091

(22) PCT Filed: Apr. 1, 2019
(86) PCT No.: PCT/JP2019/014417

§ 371 Date: Nov. 12, 2019

(30) Foreign Application Priority Data
Aug. 6, 2018 (JP) 2018-147827

(51) Int. Cl. A61F 13/49; A61F 13/15; D04H 3/04; D04H 3/147; D06M 17/00; B32B 5/02; B32B 5/04; B32B 5/26; B32B 7/022

(52) U.S. Cl. CPC A61F 13/49019; B32B 2262/12; A61F 13/15203; D04H 3/04; D04H 3/147; D06M 17/00; B32B 5/022; B32B 5/04; B32B 5/26; B32B 7/022; A61F 2013/15406; A61F 2013/15861; B32B 2555/02; B32B 2262/0284; B32B 2262/0253; B32B 2307/51; A61F 13/15585

(57) ABSTRACT

The present invention provides a stretch sheet (1) for an absorbent article in which a plurality of elastic filaments (4) that are arranged so as to extend in one direction without intersecting one another are joined to extensible sheet materials (2 and 3) over their entire length in a substantially non-stretched state. Some or all of the plurality of elastic filaments (4) are constricted filaments (40) having at least one narrow portion (40k) in a cross section that is orthogonal to a longitudinal direction (Y). It is preferable that the cross section of each constricted filament (40) that is orthogonal to the longitudinal direction (Y) has a shape in which a plurality of circles are connected together in a partially overlapping state, and it is preferable that the center-to-center distance between adjacent circles is shorter than the sum of the radius of a first one of the circles and the radius of a second one of the circles, and longer than the shorter radius of the radius of the first circle and the radius of the second circle.

### TECHNICAL FIELD

The present invention relates to a stretch sheet for an absorbent article and a method for producing the stretch sheet.

### BACKGROUND ART

As a stretch sheet, the applicant of the present invention has proposed a stretch sheet in which a plurality of elastic filaments that are arranged so as to extend in one direction without intersecting one another are joined to an extensible sheet material over their entire length in a substantially non-stretched state (see Patent Literature 1).

The stretch sheet disclosed in Patent Literature 1 is produced by performing steps of spinning a melted elastic resin from a plurality of spinning nozzles to obtain a plurality of elastic filaments in a melted or softened state, taking up and drawing the plurality of elastic filaments at a predetermined rate, and fusion-bonding the elastic filaments to a sheet material before the elastic filaments solidify.

Moreover, with regard to a method for producing such a stretch sheet, the applicant of the present invention has proposed a method for producing a stretch sheet using a spinning head that has a staggered arrangement portion in which a plurality of spinning nozzles are arranged in a so-called staggered pattern (see Patent Literature 2). Patent Literature 2 states that a spinning head is used in which the spinning nozzles are arranged in a staggered pattern, or more specifically, a plurality of nozzle rows in each of which a plurality of spinning nozzles are arranged at predetermined intervals in one direction are arranged in a direction that is orthogonal to the arrangement direction of the spinning nozzles, and the spinning nozzles in adjacent nozzle rows in that orthogonal direction are shifted from each other.

### CITATION LIST

### Patent Literatures

Patent Literature 1: JP 2008-179128A

Patent Literature 2: JP 2017-61064A

### SUMMARY OF INVENTION

The present invention provides a stretch sheet in which a plurality of elastic filaments that are arranged so as to extend in one direction without intersecting one another are joined to an extensible sheet material over their entire length in a substantially non-stretched state. In exemplary embodiments, the stretch sheet is a stretch sheet for an absorbent article. In exemplary embodiments, some or all of the elastic filaments are constricted filaments having at least one narrow portion in a cross section that is orthogonal to an extending direction in which the elastic filaments extend.

Also, the present invention provides a method for producing a stretch sheet, the method including a fusion-bonding step of bringing a plurality of elastic filaments in a melted or softened state discharged from a plurality of spinning nozzles into contact with a raw material sheet of a sheet material before the elastic filaments solidify, to thereby fusion-bond the elastic filaments to the raw material sheet. In exemplary embodiments, the stretch sheet is a stretch sheet for an absorbent article. In exemplary embodiments, in the fusion-bonding step, a plurality of nozzle rows in each of which a plurality of spinning nozzles of the spinning nozzles are arranged at intervals in a first direction are formed in a second direction that is orthogonal to the first direction. In exemplary embodiments, a spinning head is used in which the positions of the spinning nozzles in the nozzle rows that are adjacent to each other in the second direction are shifted from each other in the first direction. In exemplary embodiments, a take-up means that takes up elastic filaments spun from the spinning nozzles of the spinning head is used. In exemplary embodiments, the elastic filaments in a melted or softened state spun from the spinning nozzles are taken up at a take-up rate from 40 m/min to 200 m/min.

### BRIEF DESCRIPTION OF DRAWINGS

FIG. 1(a) is a perspective view showing a stretched state of a stretch sheet according to a preferred embodiment of the present invention, and FIG. 1(b) is a perspective view schematically showing a state in which the stretch sheet is disjoined into two sheet materials and a plurality of elastic filaments.

FIG. 2 is a cross-sectional view schematically showing a cross section of the stretch sheet of the present embodiment (cross-sectional view of a portion corresponding to an A-A cross section of the stretch sheet shown in FIG. 1).

FIG. 3(a) is a front view schematically showing a single filament shown in FIG. 2, FIG. 3(b) is a front view schematically showing a constricted filament having one narrow portion shown in FIG. 2, and FIG. 3(c) is a front view schematically showing a constricted filament having two narrow portions shown in FIG. 2.

FIG. 4 is a perspective view schematically showing relevant portions of a spinning apparatus that is used to perform a fusion-bonding step of a method for producing the stretch sheet of the present embodiment.

FIG. 5 is a perspective view schematically showing a spinning head of the spinning apparatus shown in FIG. 4, with a lower end surface (nozzle installation surface) side of the spinning head facing upward.

FIG. 6 is a plan view schematically showing the lower end surface of the spinning bed shown in FIG. 5.

FIG. 7 is a perspective view schematically showing relevant portions of a drawing apparatus that is used to perform a stretchability imparting step of the method for producing the stretch sheet of the present embodiment.

### DESCRIPTION OF EMBODIMENTS

The above-described stretch sheet is used for various uses, and the need to adjust stress may arise depending on the use or the like. In the case where the stress in the stretch sheet is to be improved, if, for example, the number of elastic filaments joined to the sheet materials is increased, the area of regions in the stretch sheet where the elastic filaments are not present decreases. Since the above-described stretch sheet is obtained by fusion-bonding elastic filaments obtained by drawing an elastic resin in a melted or softened state to the sheet materials, if the area of the regions where the elastic filaments are not present, that is, the area of the regions where the elastic filaments are not joined decreases, the thickness of the stretch sheet decreases. Therefore, there is room for improvement in tactile feel.

If, for example, improvement of the stress in the stretch sheet is attempted by increasing the diameter of the elastic filaments, the area of contact between the elastic filaments and the sheet materials increases, and, for example, a hole or the like may be formed in the sheet materials during processing for imparting stretchability to the sheet materials. Therefore, there is room for improvement in appearance.

The inventors of the present invention found that in the case where stress is to be improved, a stretch sheet in which stress can be improved while having a favorable tactile feel and without the appearance being impaired can be obtained by using elastic filaments having at least one narrow portion in a cross section that is orthogonal to the extending direction in which the elastic filaments extend. Moreover, the inventors of the present invention found that elastic filaments having such narrow portions can be efficiently obtained by arranging spinning nozzles in a staggered pattern and setting the elastic filament take-up rate to be within a predetermined rate range. In Patent Literature 1 and Patent Literature 2, there is no description to the effect that a stretch sheet includes such elastic filaments having a narrow portion and that the resin spinning rate and take-up rate of elastic filaments are set to be within predetermined rate ranges.

Therefore, the present invention relates to a stretch sheet that can eliminate the above-described drawbacks of the related art, and a method for producing the stretch sheet.

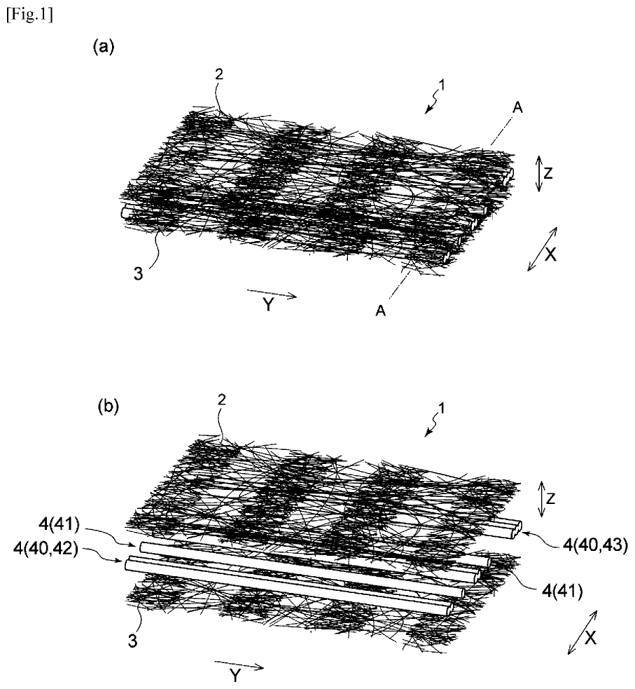

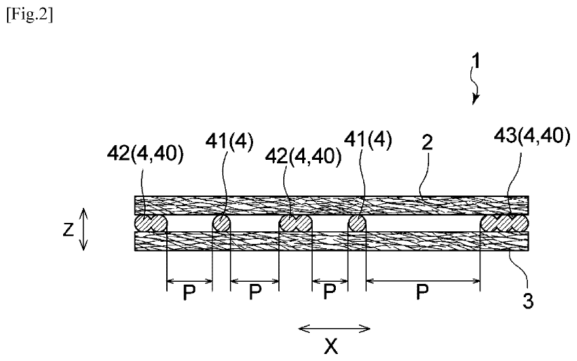

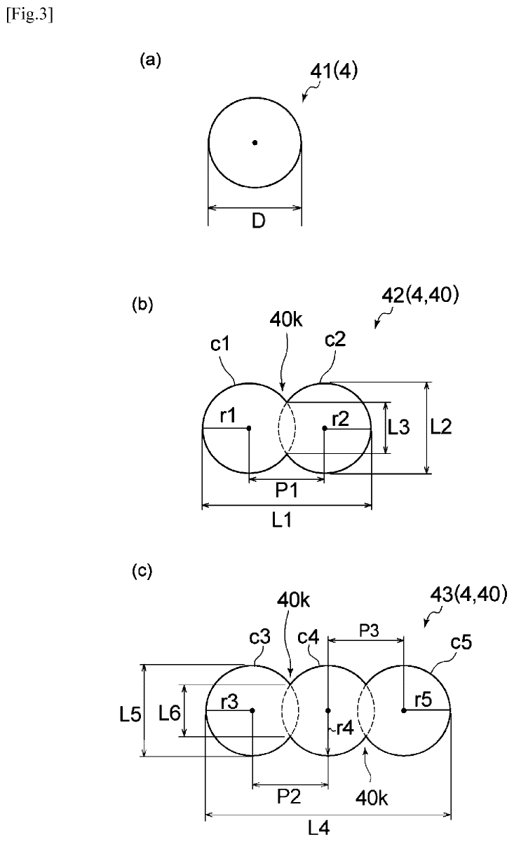

Hereinafter, the present invention will be described based on a preferred embodiment thereof, with reference to the drawings. FIGS. 1(a), 1(b), and 2 show a stretch sheet 1 according to a preferred embodiment of the present invention, and FIGS. 3(a) to 3(c) show elastic filaments 4 included in the stretch sheet 1. The stretch sheet is preferably a stretch sheet for an absorbent article.

As shown in FIGS. 1(a), 1(b), and 2, the stretch sheet 1 has a configuration in which a plurality of filamentous elastic filaments 4 are joined to two sheet materials 2 and 3, and the plurality of elastic filaments 4 are arranged so as to extend in one direction without intersecting one another. Each of the plurality of elastic filaments 4 is sandwiched between the two sheet materials 2 and 3, and, in this state, joined to the two sheet materials 2 and 3 over the entire length in one direction (longitudinal direction indicated by a reference symbol Y) of the stretch sheet 1 in a substantially non-stretched state. As used herein, “elastic” means the property of being able to be stretched and contracting when released from the stretching force, and the “substantially non-stretched state” means a state of the elastic filaments in which the elastic filaments do not contract upon removal of external force.

The stretch sheet 1 realizes stretchability due to the elasticity of the elastic filaments 4. When the stretch sheet 1 is stretched in the same direction as the extending direction of the elastic filaments 4, the elastic filaments 4 and the sheet materials 2 and 3 are stretched. Then, when stretching of the stretch sheet 1 is cancelled, the elastic filaments 4 contract, and, in accordance with this contraction, the sheet materials 2 and 3 return to the pre-stretching state. Since no other elastic filaments bound to the elastic filaments 4 while extending orthogonally thereto are present in the stretch sheet 1, when the stretch sheet 1 is stretched in the same direction as the extending direction of the elastic filaments 4, the stretch sheet 1 is stretched substantially without causing so-called “width shrinkage”, that is, shrinkage in the direction that is orthogonal to the stretching direction.

Depending on the specific use, the basis weight of the entire stretch sheet 1 is preferably 10 g/m² or greater, and more preferably 20 g/m² or greater, is preferably 80 g/m² or less, and more preferably 70 g/m² or less, and, specifically, is preferably from 10 g/m² to 80 g/m² and more preferably from 20 g/m² to 70 g/m².

From the viewpoint of realizing a favorable tactile feel, the thickness of the stretch sheet 1 is preferably 0.32 mm or greater, more preferably 0.36 mm or greater, and even more preferably 0.39 mm or greater, is preferably 0.5 mm or less, and more preferably 0.4 mm or less, and, specifically, is preferably from 0.32 mm to 0.5 mm, more preferably from 0.36 mm to 0.5 mm, and even more preferably from 0.39 mm to 0.4 mm. Measurement of the thickness of the stretch sheet 1 is performed in the following manner. The thickness of a sheet to be measured can be measured by sandwiching the sheet to be measured between flat plates under a load of 0.5 cN/cm² and measuring the distance between the flat plates.

From the viewpoint of preventing formation of a hole or the like when imparting stretchability to a composite sheet 1′ in a stretchability imparting step, which will be described later, the peeling strength of the stretch sheet 1 (peeling strength of each of the sheet materials 2 and 3 with respect to the elastic filaments 4) is preferably 5 cN/filament or greater, and more preferably 10 cN/filament or greater, is preferably 30 cN/filament or less, more preferably 20 cN/filament or less, and even more preferably 18 cN/filament or less, and, specifically, is preferably from 5 cN/filament to 30 cN/filament, more preferably from 10 cN/filament to 20 cN/filament, and even more preferably from 10 cN/filament to 18 cN/filament. Measurement of the peeling strength of the stretch sheet 1 is performed in the following manner. Each of the two sheet materials 2 and 3 of the stretch sheet 1, which is the sheet to be measured, is held in a chuck, and the sheet material 2 of the two sheet materials is peeled apart at a rate of 300 mm/min. The peeling strength of the stretch sheet 1 can be measured by measuring the maximum load at this time.

From the viewpoint of realizing sufficiently stretchable characteristics, it is preferable that the ratio (50% return load/50% stretch load) of the load (hereinafter also referred to as “50% return load”) of the stretch sheet 1 when stretched by 100% along the extending direction of the elastic filaments 4 and returned by 50% from that state to the load (hereinafter also referred to as “50% stretch load”) of the stretch sheet 1 when stretched by 50% along the extending direction of the elastic filaments 4 is 45% or greater, and preferably 50% or greater, and is preferably 100% or less. Specifically, the above-described ratio is preferably from 45% to 100%, and more preferably from 50% to 100%.

From the same viewpoint, the 50% return load is preferably 80 cN/50 mm or greater, and more preferably 120 cN/50 mm or greater, is preferably 150 cN/50 mm or less, and more preferably 135 cN/50 mm or less, and, specifically, is preferably from 80 cN/50 mm to 150 cN/50 mm, and more preferably from 120 cN/50 mm to 135 cN/50 mm.

From the same viewpoint, the 50% stretch load is preferably 80 cN/50 mm or greater, more preferably 120 cN/50 mm or greater, and even more preferably 245 cN/50 mm or greater, is preferably 600 cN/50 mm or less, more preferably 300 cN/50 mm or less, and even more preferably 250 cN/50 mm or less, and, specifically, is preferably from 80 cN/50 mm to 600 cN/50 mm, more preferably from 120 cN/50 mm to 300 cN/50 mm, and even more preferably from 245 cN/50 mm to 250 cN/50 mm.

### Method for Measuring 50% Return Load and 50% Stretch Load:

A 100% elongation cycle test of a stretch sheet is performed using a tensile tester (AG-IS manufactured by Shimadzu Corporation). Specifically, first, a sample of a stretch sheet for use in the 100% elongation cycle test is prepared, and the sample is attached to the tensile tester such that the tensile direction coincides with the extending direction in which the elastic filaments extend. At this time, the distance between chucks is set to be 150 mm. The sample is stretched 150 mm (the distance between chucks becomes 300 mm in total) at a rate of 300 mm/min in a stretching/contracting direction of the sample, and immediately after that, the sample is restored to its initial length at a rate of 300 mm/min. Note that the initial length between the chucks can be changed depending on the length of the sample to be tested.

In the 100% elongation cycle test, an elongation of 100% refers to a state in which the sample is stretched to twice the initial length. In the course of stretching the sample to an elongation of 100%, the tensile force at the point in time when the elongation is 50%, that is, the tensile force at the point in time when the sample is stretched to 1.5 times the initial length is referred to as the “50% stretch load”, and in the course of returning the sample to its initial length after stretching the sample to an elongation of 100%, the tensile force at the point in time when the elongation is 50% is referred to as the “50% return load”.

In the case where a stretch sheet incorporated in an absorbent article such as a commercially-available diaper is to be measured, an adhesive used in the absorbent article is dissolved in an organic solvent, and the stretch sheet is taken out. An organic solvent that does not dissolve an elastic body is employed as the organic solvent that is used at this time. Then, the stretch sheet that has been taken out is dried, and after that, the stretch sheet is subjected to measurement using the above-described measurement method. With respect to the measurement of other properties described in this specification as well, the stretch sheet that has been taken out as described above is subjected to the measurement after being dried.

Both of the two sheet materials 2 and 3 constituting the stretch sheet 1 are extensible. The two sheet materials 2 and 3 contain substantially non-elastic fibers, or typically contain non-elastic fibers, and are substantially non-elastic, or typically are non-elastic. Each of the sheet materials 2 and 3 is extensible in the same direction as the extending direction (longitudinal direction indicated by the reference symbol Y in FIG. 1) of the elastic filaments 4. As used herein, “extensible” encompasses (a) a case where constituent fibers themselves of the sheet materials 2 and 3 are extensible; and (b) a case where, even though the constituent fibers themselves are not extensible, a nonwoven fabric as a whole is extensible as a result of fibers bound to each other at intersections being separated from each other, a three-dimensional structure formed of a plurality of fibers through binding or the like of the fibers changing structurally, constituent fibers tearing, or the slack in fibers being taken up.

Each of the plurality of elastic filaments 4 constituting the stretch sheet 1 extends substantially continuously over the entire length of the stretch sheet 1, and typically extends continuously over the entire length thereof as in the embodiment shown in FIG. 1. Each elastic filament 4 contains an elastic resin. The plurality of elastic filaments 4 are arranged so as to extend in one direction without intersecting one another in the direction that is orthogonal to the extending direction. As long as the elastic filaments 4 do not intersect one another, the elastic filaments 4 may extend linearly or may extend while meandering. The extending direction, which is the direction in which the elastic filaments extend, typically coincides with the longitudinal direction Y, and the direction that is orthogonal to the extending direction typically coincides with a width direction X.

Each elastic filament 4 is joined to the sheet materials 2 and 3 in a substantially non-stretched state. Since the elastic filaments 4 are joined to the sheet materials 2 and 3 in a non-stretched state, relaxation (creeping) of the stretch sheet 1 due to stretching thereof does not occur, and the stretch sheet 1 has the advantage of being less likely to deteriorate in terms of stretchability. Also, the stretch sheet 1 has the advantage of being capable of stretching up to the extensible length of the sheet materials 2 and 3 or the maximum elongation of the elastic filaments 4. It is preferable that the elastic filaments 4 are each fusion-bonded to the extensible sheet materials 2 and 3 over their entire length in a substantially non-stretched state. Here, the wording “fusion-bonded” does not mean that the elastic filaments 4 are joined to the sheet materials 2 and 3 via a third component such as an adhesive, but rather means that the elastic filaments 4 are joined to the sheet materials 2 and 3 as a result of at least one of the resin constituting the elastic filaments 4 and the resin constituting the sheet materials 2 and 3 being melted.

From the viewpoint of realizing a favorable tactile feel, the end-to-end distance P (see FIG. 2) of elastic filaments 4 that are adjacent to each other in the width direction X is preferably 0.4 mm or greater, and more preferably 0.6 mm or greater, is preferably 1.2 mm or less, more preferably 1 mm or less, and even more preferably 0.8 mm or less, and is preferably from 0.4 to 1.2 mm, more preferably from 0.6 to 1 mm, and even more preferably from 0.6 to 0.8 mm. The end-to-end distance P may be fixed between all of the elastic filaments, or, as shown in FIG. 2, the end-to-end distance P between a pair of elastic filaments may be different from that between another pair of elastic filaments. In the case where the end-to-end distance P is not fixed, it is preferable that an average value of the end-to-end distances between elastic filaments is within the above-described preferred range, and it is more preferable that the end-to-end distances between all of the elastic filaments are within the above-described preferred range.

### Method for Measuring End-to-End Distance Between Adjacent Elastic Filaments:

To measure the end-to-end distance P between elastic filaments 4 that are adjacent to each other, a cut surface of a sample of a stretch sheet that is cut along the width direction is magnified under a microscope. The measurement is performed at 100 random points, and the thus obtained average value is used as the average value of the end-to-end distances. The measurement is performed by cutting a plurality of cut surfaces at different positions in the longitudinal direction, and measuring the end-to-end distance between adjacent elastic filaments in each of the plurality of cut surfaces.

Some elastic filaments of the elastic filaments 4 included in the stretch sheet 1 are constricted filaments 40 each having at least one narrow portion 40k in a cross section (hereinafter also referred to as “cross section in the width direction”) that is orthogonal to the extending direction (longitudinal direction Y), in which the elastic filaments extend. The elastic filaments 4 include single filaments 41 each having no narrow portion 40k and constricted filaments 40 each having at least one narrow portion 40k. Here, a filament having one narrow portion in a cross section that is orthogonal to the extending direction means that when the cross section that is orthogonal to the extending direction of the filament is viewed, there is a pair of depressed portions that are depressed from a peripheral surface of the filament toward the inner side of the cross section. For example, having two narrow portions means that there are two pairs of such depressed portions. More specifically, in the embodiment shown in FIG. 2, the constricted filaments 40 include filaments 42 each having one narrow portion 40k and a filament 43 having two narrow portions 40k. Note that all of the elastic filaments 4 included in the stretch sheet for an absorbent article of the present invention may be constricted filaments 40 each having at least one narrow portion.

The single filaments 41 are obtained by drawing a melted resin discharged from spinning nozzles 12, which will be described later, on a spinning line. As shown in FIG. 3(a), the diameter D of a single filament 41 is not particularly limited, but in light of balance between the texture of the stretch sheet 1 and the productivity of the elastic filaments 4, the diameter D is preferably 40 μm or greater, and more preferably 80 μm or greater, is preferably 200 μm or less, and more preferably 180 μm or less, and is from 40 μm to 200 μm, and more preferably from 80 μm to 180 μm. The diameter of the single filaments 41 depends on the diameter of the spinning nozzles 12, which will be described later.

The constricted filaments 40 are obtained by, for example, two or more single filaments discharged from two or more adjacent spinning nozzles 12 binding together in the course of drawing. A cross section in the width direction of the constricted filaments 40 has a shape in which a plurality of circles are connected together in the width direction X in a partially overlapping state. Specifically, a cross section in the width direction of the filaments 42 having one narrow portion has a shape in which, as shown in FIG. 3(b), two circles c1 and c2 are connected to each other in the width direction X in a partially overlapping state. A cross section in the width direction of the filaments 43 having two narrow portions has a shape in which, as shown in FIG. 3(c), three circles c3, c4, and c5 are connected together in the width direction X in a partially overlapping state. The overlapping portions of the circles that are connected together are the portions that are located at connecting portions between adjacent circles and enclosed by circular arcs shown in dashed lines in FIGS. 3(b) and 3(c).

As a result of making a cross section in the width direction have a shape in which a plurality of circles are connected together in a partially overlapping state, that is, providing at least one narrow portion 40k in the cross section in the width direction, it is possible to improve the stress in the stretch sheet 1 while suppressing a reduction in the end-to-end distance between adjacent elastic filaments 4, unlike a case where the number of single filaments 41 is increased, for example. That is to say, it is possible to improve the stress in the stretch sheet 1 while achieving a favorable tactile feel by maintaining a large thickness of the stretch sheet 1. Compared with a case where the cross-sectional area of single filaments 41 is increased by increasing the diameter thereof, an increase in the area of contact between the sheet materials 2 and 3 and the elastic filaments 4 can be suppressed. That is to say, while improving the stress in the stretch sheet 1 by increasing the cross-sectional area of the elastic filaments 4, it is possible to maintain a favorable appearance by suppressing the formation of a hole or the like in the stretchability imparting step, which will be described later. As described above, with the stretch sheet of the present embodiment, it is possible to improve the stress produced by the elastic filaments 4 while achieving a favorable tactile feel of the stretch sheet and maintaining a favorable appearance thereof.

As shown in FIG. 1(b), a narrow portion 40k of a constricted filament 40 extends along the extending direction (longitudinal direction Y) of the elastic filaments 4. As a result of the constricted filaments 40 of the stretch sheet 1 having the narrow portions 40k extending along the extending direction of the elastic filaments 4, it is possible to further improve the stress produced by the elastic filaments 4 while achieving a favorable tactile feel of the stretch sheet and maintaining a favorable appearance thereof.

From the viewpoint of realizing a favorable tactile feel and maintaining a favorable appearance, it is preferable that in an external shape of a cross section in the width direction of each constricted filament 40, the center-to-center distance P1 between adjacent circles is smaller than the sum of the radius of a first one of the circles and the radius of a second one of the circles, and longer than the shorter radius of the radius of the first circle and the radius of the second circle. Specifically, as shown in FIG. 3(b), in an external shape of a cross section in the width direction of a filament 42 having one narrow portion, it is preferable that the center-to-center distance P1 between adjacent circles c1 and c2 is smaller than the sum (r1+r2) of the radius r1 of the first circle c1 and the radius r2 of the second circle c2, and longer than the shorter radius (e.g., the radius r1 of the first circle c1) selected from the radius r1 of the first circle c1 and the radius r2 of the second circle c2. Preferably, the radius r1 of the circle c1 and the radius r2 of the circle c2 are radii in a direction that is the same as the width direction X.

Similarly, as shown in FIG. 3(c), in a cross section in the width direction of a filament 43 having two narrow portions, it is preferable that the center-to-center distance P2 between adjacent circles c3 and c4 is smaller than the sum (r3+r4) of the radius r3 of the first circle c3 and the radius r4 of the second circle c4, and longer than the shorter radius (e.g., the radius r3 of the first circle c3) selected from the radius r3 of the first circle c3 and the radius r4 of the second circle c4. Moreover, it is preferable that the center-to-center distance P3 between adjacent circles c4 and c5 is smaller than the sum (r4+r5) of the radius r4 of the first circle c4 and the radius r5 of the second circle c5, and longer than the shorter radius (e.g., the radius r4 of the first circle c4) selected from the radius r4 of the first circle c4 and the radius r5 of the second circle c5. It is preferable that the radius r3 of the circle c3 and the radius r5 of the circle c5 are radii in a direction that is the same direction as the width direction, and it is preferable that the radius r4 of the circle c4 is a radius in a direction that is the same as the thickness direction Z.

In a cross section in the width direction (cross section that is orthogonal to the extending direction) of a constricted filament 40, the maximum length of the constricted filament in the width direction X is preferably 100 μm or greater, and more preferably 200 μm or greater, is preferably 800 μm or less, more preferably 400 μm or less, even more preferably 500 μm or less, and especially preferably 270 μm or less, and, specifically, is preferably from 100 μm to 800 μm, more preferably from 100 μm to 400 μm, even more preferably from 200 μm to 500 μm, and especially preferably from 200 μm to 270 μm. The maximum length of the constricted filament in the width direction X is the maximum length in the width direction of a cross section in the width direction of the constricted filament, and can be measured using the following method.

### Method for Measuring the Maximum Length of Constricted Filament in Width Direction:

To measure the maximum length of the constricted filament 40 in the width direction X, a cut surface of a sample of a stretch sheet when cut along the width direction is magnified under a microscope. The measurement is performed at 30 random points for each constricted filament, the 30 random points being located at different positions in the longitudinal direction, and the average value thereof is used as the maximum length of the constricted filament 40 in the width direction.

From the viewpoint of realizing a favorable tactile feel, as shown in FIG. 3(b), in a filament 42 having one narrow portion, the ratio ((L2/L1)×100) of the length L2 in the thickness direction Z to the length L1 in the width direction X is preferably 10% or greater, and more preferably 30% or greater, is preferably 60% or less, and more preferably 50% or less, and, specifically, is preferably from 10% to 60%, and more preferably from 30% to 50%. Note that the length L1 in the width direction X of a filament 42 having one narrow portion means the maximum length in the width direction X, and the length L2 in the thickness direction Z of a filament 42 having one narrow portion means the maximum length, that is, the diameter in the thickness direction Z.

From the same viewpoint, in a filament 42 having one narrow portion, the ratio ((L3/L2)×100) of the minimum length L3 of the narrow portion 40k in the thickness direction Z to the length L2 in the thickness direction Z is preferably 5% or greater, and more preferably 10% or greater, is preferably 50% or less, and more preferably 30% or less, and, specifically, is preferably from 5% to 50%, and more preferably from 10% to 30%.

From the same viewpoint, the maximum length L1 in the width direction of a filament 42 having one narrow portion is preferably 100 μm or greater, and more preferably 200 μm or greater, is preferably 400 μm or less, and more preferably 300 μm or less, and, specifically, is preferably from 100 μm to 400 μm, and more preferably from 200 μm to 300 μm.

From the same viewpoint, the maximum length L2 in the thickness direction of a constricted filament 40 is preferably 80 μm or greater, and more preferably 100 μm or greater, is preferably 200 μm or less, and more preferably 180 μm or less, and, specifically, is preferably from 80 μm to 200 μm, and more preferably from 100 μm to 180 μm.

From the same viewpoint, the minimum length L3 in the thickness direction Z of a narrow portion 40k of a constricted filament 40 is preferably 5 μm or greater, and more preferably 10 μm or greater, is preferably 60 μm or less, and more preferably 50 μm or less, and, specifically, is preferably from 5 μm to 60 μm, and more preferably from 10 μm to 50 μm.

From the viewpoint of realizing a favorable tactile feel, as shown in FIG. 3(c), in a filament 43 having two narrow portions, the ratio ((L5/L4)×100) of the length L5 in the thickness direction Z to the length L4 in the width direction X is preferably 5% or greater, and more preferably 10% or greater, is preferably 60% or less, and more preferably 50% or less, and, specifically, is preferably from 5% to 60%, and more preferably from 10% to 50%. Note that the length L4 in the width direction X of a filament 43 having two narrow portions means the maximum length in the width direction X, and the length L5 in the thickness direction Z of a filament 43 having two narrow portions means the maximum length in the thickness direction Z.

From the same viewpoint, in a filament 43 having two narrow portions, the ratio ((L6/L5)×100) of the minimum length L6 of the narrow portion 40k in the thickness direction Z to the length L5 in the thickness direction Z is preferably 5% or greater, and more preferably 10% or greater, is preferably 50% or less, and more preferably 30% or less, and, specifically, is preferably from 5% to 50%, and more preferably from 10% to 30%.

From the same viewpoint, the maximum length L4 in the width direction of a filament 43 having two narrow portions is preferably 200 μm or greater, and more preferably 300 μm or greater, is preferably 600 μm or less, and more preferably 500 μm or less, and, specifically, is preferably from 200 μm to 600 μm, and more preferably from 300 μm to 500 μm.

From the same viewpoint, the maximum length L5 in the thickness direction of a filament 43 having two narrow portions is preferably 80 μm or greater, and more preferably 100 μm or greater, is preferably 200 μm or less, and more preferably 180 μm or less, and, specifically, is preferably from 80 μm to 200 μm, and more preferably from 100 μm to 180 μm.

From the same viewpoint, the minimum length L6 in the thickness direction Z of a narrow portion 40k of a filament 43 having two narrow portions is preferably 5 μm or greater, and more preferably 10 μm or greater, is preferably 60 μm or less, and more preferably 50 μm or less, and, specifically, is preferably from 5 μm to 60 μm, and more preferably from 10 μm to 50 μm.

In the stretch sheet 1, from the viewpoint of realizing a favorable tactile feel and suppressing deterioration of appearance, the ratio ((number of constricted filaments 40/number of elastic filaments 4)×100) of the number of constricted filaments 40 to the number of elastic filaments 4 is preferably 5% or greater, more preferably 20% or greater, and even more preferably 50% or greater, is preferably 90% or less, more preferably 80% or less, and even more preferably 70% or less, and, specifically, is preferably from 5% to 90%, more preferably from 20% to 80%, and even more preferably from 50% to 70%. Note that the constricted filaments 40 of the embodiment shown in FIG. 2 and the like are each formed by a plurality of filaments adhering to each other immediately after being spun; however, to count the “number of elastic filaments”, a filament that is incorporated in the stretch sheet 1 while remaining in an immediate post-spinning state, a filament 42 having one narrow portion, and a filament 43 having two narrow portions are each counted as “one elastic filament”.

From the same viewpoint, in the case where the plurality of elastic filaments 4 include a plurality of constricted filaments 40, the ratio ((number of filaments 42 having one narrow portion/number of constricted filaments 40)×100) of the number of filaments 42 having one narrow portion to the number of constricted filaments 40 is preferably 50% or greater, more preferably 60% or greater, and even more preferably 90% or greater, is preferably 100% or less, and, specifically, is preferably from 50% to 100%, more preferably from 60% to 100%, and even more preferably from 90% to 100%.

Materials that form the stretch sheet 1 is described. For the sheet materials 2 and 3, for example, nonwoven fabrics such as an air-through nonwoven fabric, a heat-rolled nonwoven fabric, a spunlaced nonwoven fabric, a spunbonded nonwoven fabric, and a melt-blown nonwoven fabric can be used. These nonwoven fabrics may be composed of continuous filaments or staple fibers. The sheet materials 2 and 3 may be of the same type or different types. As used herein, the “sheet materials of the same type” means sheet materials that are identical with respect to all of the sheet material production process, the type of constituent fibers of the sheet materials, the fiber diameter and length of the constituent fibers, the thickness and the basis weight of the sheet materials, and the like. If the sheet materials are different with respect to at least one of these, the sheet materials are the “sheet materials of different types”.

The thickness of the sheet materials 2 and 3 is preferably 0.05 mm or greater, more preferably 0.1 mm or greater, and even more preferably 0.15 mm or greater, is preferably 5 mm or less, more preferably 1 mm or less, and even more preferably 0.5 mm or less, and, specifically, is preferably from 0.05 mm to 5 mm, more preferably from 0.1 mm to 1 mm, and even more preferably from 0.15 mm to 0.5 mm. To measure the thickness, a cross section of the stretch sheet 1 sandwiched between flat plates under a load of 0.5 cN/cm² is observed under a microscope at a magnification of 50 to 200 times, the average thickness is obtained in each visual field, and an average value of the average thickness values of three visual fields is obtained. The thickness of the entire sheet can be obtained by measuring the distance between the flat plates. In light of the texture, thickness, design quality, and the like, the basis weight of each of the sheet materials 2 and 3 is preferably 3 g/m² or greater, and more preferably 5 g/m² or greater, is preferably 100 g/m² or less, and more preferably 30 g/m² or less, and, specifically, is preferably from 3 g/m² to 100 g/m², and more preferably from 5 g/m² to 30 g/m².

For example, non-elastic fibers that are substantially non-elastic can be used as the constituent fibers of the sheet materials 2 and 3, and in that case, the sheet materials 2 and 3 may constitute extensible fiber layers mainly composed of the non-elastic fibers. Examples of such non-elastic fibers include fibers and the like made of polyesters, such as polyethylene (PE), polypropylene (PP), polyethylene terephthalate (PET), and polybutylene terephthalate (PBT), polyamides, and the like. The constituent fibers of the sheet materials 2 and 3 may be staple fibers or filament fibers and may be hydrophilic or water-repellent. Moreover, core-sheath type or side-by-side type composite fibers, splittable fibers, modified cross-section fibers, crimped fibers, heat-shrinkable fibers, and the like can also be used. These fibers can be used alone or in a combination of two or more.

A preferred example of the constituent fibers of the sheet materials 2 and 3 is a fiber made of at least two components including a lower-melting-point component and a higher-melting-point component. In that case, the constituent fibers are joined together at fiber intersections through thermal fusion-bonding of at least the lower-melting-point component. Preferred core-sheath type composite fibers made of at least two components including a lower-melting-point component and a higher-melting-point component are those in which the core is made of high-melting-point PET or PP and the sheath is made of lower-melting-point PET, PP, or PE. The use of these composite fibers strengthens the fusion-bonding of the elastic filaments 4 to the sheet materials and makes it less likely that peeling will occur, and is therefore preferable.

The raw material of the elastic filaments 4 is an elastic resin, such as a thermoplastic elastomer or rubber, for example. When a thermoplastic elastomer is used as the raw material, melt spinning using an extruder can be performed as in the case of an ordinary thermoplastic resin, and the thus obtained elastic filaments can be thermally fusion-bonded with ease and are therefore suitable for the stretch sheet 1. Examples of the thermoplastic elastomer include styrene-based elastomers, such as SBS (styrene-butadiene-styrene), SIS (styrene-isoprene-styrene), SEB S (styrene-ethylene-butadiene-styrene), and SEPS (styrene-ethylene-propylene-styrene) elastomers, olefin-based elastomers (ethylene-based α-olefin elastomers, and propylene-based elastomers in which ethylene, butene, octene, and the like are copolymerized), polyester-based elastomers, polyurethane-based elastomers, and the like, and these thermoplastic elastomers can be used alone or in a combination of two or more.

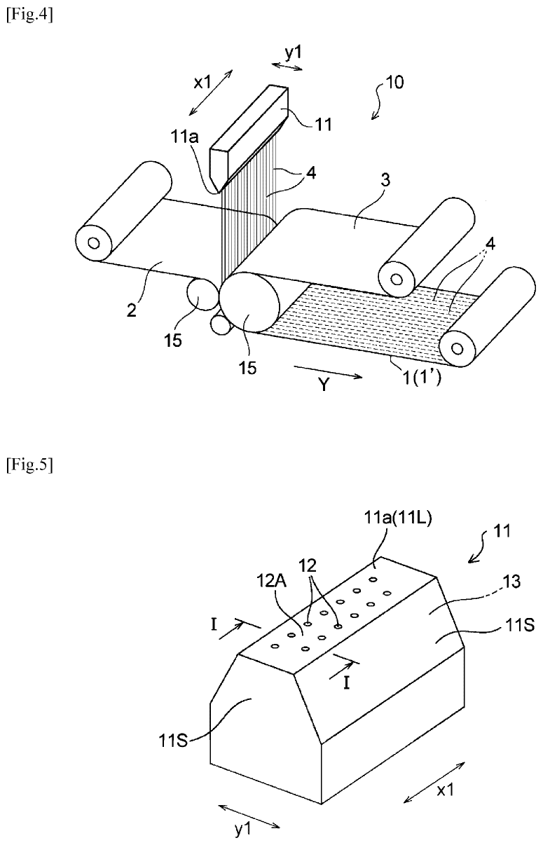

A method for producing the stretch sheet 1 of the present invention is described with reference to FIGS. 4 to 7 using a method for producing the stretch sheet 1 of the above-described embodiment as an example. FIGS. 4 to 7 show a spinning apparatus 10 and a drawing apparatus that are used in the method for producing the stretch sheet 1 according to a preferred embodiment.

As shown in FIG. 4, the spinning apparatus 10 includes a spinning head 11 that spins a melted resin from spinning nozzles to obtain elastic filaments 4 in a melted or softened state, and a pair of nip rollers 15 serving as a take-up means that takes up a plurality of elastic filaments 4 discharged from the spinning head 11. Typically, the pair of nip rollers 15 are rollers having a smooth surface. This spinning apparatus 10 is an apparatus that prevents filaments through so-called melt blowing, and includes, in addition to the spinning head 11, a melt extruder (not shown) that melts elastic resin chips and delivers the melted resin to the spinning head 11, and the like. The basic configuration of the spinning apparatus 10 is the same as that of a known melt blowing type spinning apparatus. Moreover, the spinning head 11 and the pair of nip rollers 15 are electrically connected to a control unit, which is not shown, so that the control unit can adjust the resin discharge rate of the spinning head 11 and the take-up rate of the pair of nip rollers 15.

As shown in FIG. 5, the spinning head 11 includes a bottom wall portion 11L that forms a lower end surface 11a of the head 11 and has a rectangular shape in a plan view, and side wall portions 11S that are connected to peripheral edges of the bottom wall portion 11L, and an internal space of the spinning head 11 that is defined by these wall portions 11L and 11S constitutes a storage portion 13 for a melted resin supplied from the melt extruder. A plurality of spinning nozzles 12 are formed in the lower end surface 11a of the spinning head 11, and the storage portion 13 of the spinning head 11 is in communication with the outside via the individual spinning nozzles 12. The material of the spinning head 11 can be a material similar to that of a known spinning head, and is usually a metal.

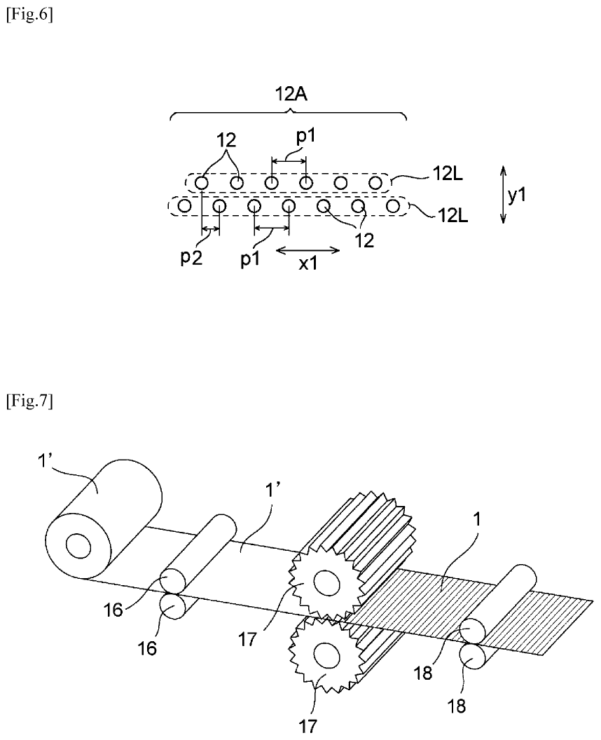

As shown in FIGS. 5 and 6, a staggered arrangement portion 12A in which the plurality of spinning nozzles 12 are arranged in a staggered pattern is provided in the lower end surface 11a, which serves as a nozzle installation surface, of the spinning head 11. In the staggered arrangement portion 12A, a plurality of (two, in the present embodiment) nozzle rows 12L (in FIG. 6, portions enclosed by dashed lines) in each of which a plurality of spinning nozzles 12 are arranged at intervals in a first direction xl of the lower end surface 11a are formed in a second direction yl that is orthogonal to the first direction xl, that is, in the width direction of the lower end surface 11a, and the positions of the spinning nozzles 12 in adjacent nozzle rows 12L, 12L are shifted from each other by half a pitch in the first direction xl. As used herein, “arranged in a staggered pattern” includes not only a configuration in which the plurality of spinning nozzles 12 are arranged exactly as described above but also a configuration in which unintentional slight displacement in the arrangement, such as displacement that is unavoidable in production, occurs.

The spinning nozzles 12 of the spinning head 11 have a circular shape in a plan view; however, in the present invention, the shape of the spinning nozzles in a plan view is not particularly limited, and can be any shape such as a polygonal shape. The diameter of the spinning nozzles 12, which have a circular shape in a plan view, affects the diameter and the drawing factor of the elastic filaments 4. From this viewpoint, the diameter of the spinning nozzles 12 is preferably 0.1 mm or greater, and more preferably 0.2 mm or greater, is preferably 2 mm or less, and more preferably 0.6 mm or less, and, specifically, is preferably from 0.1 mm to 2 mm, and more preferably from 0.2 mm to 0.6 mm.

In the case where the diameter of the spinning nozzles 12 is within the above-described range, the center-to-center distance (pitch p1) in the first direction xl between adjacent spinning nozzles 12, 12 in each nozzle row 12L of the staggered arrangement portion 12A is, from the viewpoint of developing stress, preferably 0.5 mm or greater, and more preferably 0.8 mm or greater, is preferably 2 mm or less, and more preferably 1.5 mm or less, and, specifically, is preferably from 0.5 mm to 2 mm, and more preferably from 0.8 mm to 1.5 mm. In each of the nozzle rows 12L, all of the spinning nozzles 12 are arranged with equal pitch.

In the case where the diameter of the spinning nozzles 12 is within the above-described range, the center-to-center distance (pitch p2) in the first direction xl between any single spinning nozzle 12 (hereinafter referred to as a particular nozzle 12) in one nozzle row 12L of adjacent nozzle rows 12L, 12L in the second direction yl and a spinning nozzle 12 that is the closest to that particular nozzle 12 in the other nozzle row 12L is, in light of the tactile feel, preferably 0.3 mm or greater, and more preferably 0.5 mm or greater, is preferably 1 mm or less, and more preferably 0.8 mm or less, and, specifically, is preferably from 0.3 mm to 1 mm, and more preferably from 0.5 mm to 0.8 mm.

As shown in FIG. 4, the method for producing the stretch sheet 1 of the present embodiment includes a fusion-bonding step of taking up and drawing a plurality of elastic filaments 4 in a melted or softened state discharged from the plurality of spinning nozzles 12 and fusion-bonding the elastic filaments 4 to raw material sheets 2′ and 3′ of the sheet materials 2 and 3 before the elastic filaments 4 solidify. As shown in FIG. 7, the production method includes a stretchability imparting step of subjecting a composite sheet 1′ obtained through the fusion-bonding step to drawing processing.

First, chips of an elastic resin, which is a raw material of the elastic filaments 4, are melted and kneaded using a melt extruder, which is not shown, connected to the spinning head 11, and the elastic resin in the melted state is supplied to the storage portion 13 (see FIG. 5) in the spinning head 11. As shown in FIG. 4, the thus supplied elastic resin in the melted state is discharged from the plurality of spinning nozzles 12, which are formed in the lower end surface of the spinning head 11, as the elastic filaments 4 in a melted or softened state at a certain resin discharge rate V1. Since the plurality of spinning nozzles 12 are arranged in a staggered pattern as described above, the plurality of elastic filaments 4 spun from the respective spinning nozzles 12 extend while still maintaining the form of single elastic filaments 4 without intersecting one another, and then, some adjacent single elastic filaments 4 bind to each other on the way to the position at which the filaments 4 merge with the raw material sheets 2′ and 3′ of the sheet materials 2 and 3. The adjacent single elastic filaments 4 are cooled to a certain extent on the upstream side, and then bind to each other while being cooled on the downstream side.

As shown in FIG. 4, the discharged plurality of elastic filaments 4 in the melted or softened state merge with the raw material sheets 2′ and 3′ that are unwound from raw material rolls at the same rate, and are sandwiched between the two raw material sheets 2′ and 3′ and taken up by the pair of nip rollers 15, 15 at a certain take-up rate V2.

The resin discharge rate V1 and the take-up rate V2 of the elastic filaments 4 affect the diameter and the drawing factor of the elastic filaments 4 as well as the binding properties of adjacent single elastic filaments 4. In order to efficiently produce the constricted filaments 40 shown in FIGS. 2 and 3, it is naturally necessary to use the above-described spinning head 11, and in addition to that, it is effective to adjust the resin discharge rate V1 and the take-up rate V2 of the elastic filaments 4. If the plurality of spinning nozzles 12 of the spinning head 11 are linearly arranged in a single row like a conventional configuration, there are cases where adjacent single elastic filaments 4 bind to each other on the upstream side, that is, in a highly melted state immediately after being discharged from the spinning nozzles, and form elastic filaments having a large diameter. In such cases, the area of contact between the elastic filaments and the raw material sheets 2′ and 3′ increases, and therefore, there is room for improvement with respect to the formation of a hole or the like. If just the spinning head 11 in which the spinning nozzles 12 are arranged in a staggered pattern is used, the elastic filaments 4 extend while still maintaining the form of single elastic filaments 4, and, in many cases, no constricted filaments 40 are formed. However, as in the present embodiment, by using the spinning head 11 in which the plurality of spinning nozzles 12 are arranged in a staggered pattern and adjusting the take-up rate V2, in particular, the resin discharge rate V1 and the take-up rate V2 to the following rates, the constricted filaments 40 can be efficiently produced.

From the viewpoint of efficiently producing the constricted filaments 40, the ratio ((V2/V1)×100) of the take-up rate V2 of the pair of nip rollers 15, 15 to the resin discharge rate V1 of the spinning head 11 is preferably 500% or greater, and more preferably 1,000% or greater, and is preferably 2,500% or less, and more preferably 2,000% or less.

From the same viewpoint, the resin discharge rate V1 of the spinning head 11 is preferably 5 m/min or greater, and more preferably 8 m/min or greater, is preferably 30 m/min or less, and more preferably 25 m/min or less, and, specifically, is preferably from 5 m/min to 30 m/min, and more preferably from 8 m/min to 25 m/min.

From the same viewpoint, the take-up rate V2 of the pair of nip rollers 15, 15 is preferably 40 m/min or greater, and more preferably 70 m/min or greater, is preferably 200 m/min or less, and more preferably 180 m/min or less, and, specifically, is preferably from 40 m/min to 200 m/min, and more preferably from 70 m/min to 180 m/min.

In order to efficiently produce the filaments 42 having one narrow portion, the take-up rate V2 of the pair of nip rollers 15, 15 is preferably 50 m/min or greater, and more preferably 70 m/min or greater, is preferably 180 m/min or less, and more preferably 150 m/min or less, and, specifically, is preferably from 50 m/min to 180 m/min, and more preferably from 70 m/min to 150 m/min.

The elastic filaments 4 in the melted or softened state merge with the raw material sheets 2′ and 3′ before solidifying, that is, in a fusion-bondable state. As a result, the elastic filaments 4 are fusion-bonded to these raw material sheets 2′ and 3′ in a state in which the elastic filaments 4 are sandwiched between the raw material sheets 2′ and 3′. That is to say, the elastic filaments 4 prior to solidification are fusion-bonded to the raw material sheets 2′ and 3′ that are being conveyed, and thus, the elastic filaments 4 are taken up and drawn. During fusion-bonding of the elastic filaments 4, external heat is not applied to the raw material sheets 2′ and 3′. That is to say, only the melting heat caused by the elastic filaments 4 in the fusion-bondable state allows the elastic filaments 4 to be fusion-bonded to the two raw material sheets 2′ and 3′. As a result, only the fibers that are present around the elastic filaments 4, of the constituent fibers of the two raw material sheets 2′ and 3′ are fusion-bonded to the elastic filaments 4, and the fibers that are located farther from the elastic filaments 4 than those fibers are not fusion-bonded thereto. As a result, heat that is applied to the two raw material sheets 2′ and 3′ can be minimized, and therefore, if the two raw material sheets 2′ and 3′ are, for example, nonwoven fabrics, a favorable texture that is intrinsic to the nonwoven fabrics themselves can be maintained. Thus, the resulting stretch sheet 1 has a favorable texture.

Until the spun elastic filaments 4 in the melted or softened state merge with the raw material sheets 2′ and 3′, the elastic filaments 4 are drawn, and molecules of the elastic filaments 4 are oriented in the drawing direction. The diameter of the elastic filaments 4 decreases. From the viewpoint of sufficiently drawing the elastic filaments 4 and also preventing the elastic filaments 4 from breaking, the temperature of the elastic filaments 4 may be adjusted by blowing air (hot air or cool air) at a predetermined temperature onto the spun elastic filaments 4. Drawing of the elastic filaments 4 is not limited to the drawing (melt drawing) of a resin composition (elastic resin), the resin composition forming the elastic filaments 4, in a melted state and may also be the drawing (softened drawing) of the resin composition in a softened state during a cooling process.

The temperature of the elastic filaments 4 when the elastic filaments 4 merge with the raw material sheets 2′ and 3′ is preferably 100° C. or greater, and more preferably 120° C. or greater, in order to ensure that the fibers are fusion-bonded. Moreover, from the viewpoint of keeping the shape of the elastic filaments 4 and thereby obtaining a stretch sheet 1 with favorable stretchable characteristics, the temperature of the elastic filaments 4 at the time of merging is preferably 180° C. or less, and more preferably 160° C. or less. Specifically, the temperature of the elastic filaments 4 at the time of merging is preferably from 100° C. to 180° C., and more preferably from 120° C. to 160° C. The temperature at the time of merging, that is, at the time when the elastic filaments 4 are joined to the raw material sheets 2′ and 3′ can be measured by using a film made of modified polyethylene, modified polypropylene, or the like and having a different melting point from that of the resin composition constituting the elastic filaments 4 as a laminate base material to which the elastic filaments 4 are to be joined, and observing the state of joining. At this time, if the elastic filaments 4 are fusion-bonded to the laminate base material, the joining temperature is equal to or higher than the melting point of the laminate base material.

When the elastic filaments 4 merge with (are joined to) the raw material sheets 2′ and 3′, the elastic filaments 4 are in a substantially non-stretched state (state in which the elastic filaments 4 do not contract upon removal of external force). In a state in which the elastic filaments 4 have been joined to the raw material sheets 2′ and 3′, it is more preferable that at least some of the constituent fibers of the raw material sheets 2′ and 3′ are fusion-bonded to the elastic filaments 4, or, furthermore, both the elastic filaments 4 and at least some of the constituent fibers of the raw material sheets 2′ and 3′ are fusion-bonded. The reason for this is that a sufficient joining strength can be achieved. The stretchable characteristics of the resulting stretch sheet 1 are affected by the density of joining points at which the elastic filaments 4 are joined to the raw material sheets 2′ and 3′. The stretchable characteristics not only can be adjusted using the joining temperature and the joining pressure, but can also be adjusted by drawing (see FIG. 7) the raw material sheets 2′ and 3′ through elasticity realizing processing, which will be described later. As a result of fusion-bonding the constituent fibers of the raw material sheets 2′ and 3′ to the elastic filaments 4, the joining strength at each joining point increases. It is preferable to reduce the density of joining points because stretchability inhibition by the raw material sheets 2′ and 3′ can be reduced, and a stretch sheet 1 having a sufficient joining strength can be obtained.

When the plurality of elastic filaments 4 merge with the raw material sheets 2′ and 3′, the elastic filaments 4 (single filaments 41 and constricted filaments 40) are arranged in one direction without intersecting one another. Then, in a state in which the elastic filaments 4 have merged with the raw material sheets 2′ and 3′ and the elastic filaments 4 are sandwiched between the two raw material sheets 2′ and 3′, the three members are compressed by the pair of nip rollers 15, 15. The compressing conditions affect the texture of the resulting stretch sheet 1. If the compressing force is large, the elastic filaments 4 are likely to bite into both of the raw material sheets 2′ and 3′. For this reason, in light of the texture, it is sufficient that the compressing force applied by the pair of nip rollers 15, 15 is such that the elastic filaments 4 are brought into contact with the two raw material sheets 2′ and 3′, and an excessively high compressing force is not needed. As a result of the above-described fusion-bonding step, a composite sheet 1′ in which the elastic filaments 4 are sandwiched and fixed between the two raw material sheets 2′ and 3′ is obtained.

FIG. 7 illustrates how an embodiment of the stretchability imparting step (elasticity realizing processing) is performed. The stretchability imparting step is a step in which, after the elastic filaments 4 have been fusion-bonded to the raw material sheets 2′ and 3′, the raw material sheets 2′ and 3′ are drawn in the extending direction of the elastic filaments 4, and as a result of this step, extensibility is imparted to the raw material sheets 2′ and 3′, which originally do not have extensibility. The object to be processed in the stretchability imparting step is the composite sheet 1′, which has been obtained through the fusion-bonding step shown in FIG. 4 and in which the elastic filaments 4 are fusion-bonded to the raw material sheets 2′ and 3′.

In the stretchability imparting step illustrated in FIG. 7, a drawing apparatus equipped with a pair of toothed rollers 17, 17 in each of which teeth and bottom lands are alternately formed in the circumferential direction is used. This step is performed by introducing the composite sheet 1′ between the two rollers 17, 17 and conveying the composite sheet 1′. Thus, the composite sheet 1′ is drawn in the conveyance direction, that is, the extending direction of the elastic filaments 4 and thereby formed into a stretch sheet 1 to be obtained. This drawing apparatus includes, as means for allowing the composite sheet 1′ to pass between the toothed rollers 17, 17, a pair of nip rollers 16, 16 that are arranged on the upstream side of the toothed rollers 17 in the conveyance direction of the composite sheet 1′ and a pair of nip rollers 18, 18 that are arranged on the downstream side of the toothed rollers 17 in the conveyance direction, and is configured such that the extent to which the composite sheet 1′ is drawn can be adjusted by appropriately adjusting the conveyance speed of the composite sheet 1′ using the rollers 16 and 18.

The drawing apparatus has a known lifting and lowering mechanism (not shown) that displaces a pivot portion of one or both of the pair of toothed rollers 17, 17 upward and downward, and is thus configured such that the distance between the two rollers 17, 17 can be adjusted. For example, the pair of toothed rollers 17, 17 are meshed with each other such that the teeth of a first one of the rollers 17 are inserted into the spaces between the teeth of a second one of the rollers 17 with play and the teeth of the second roller 17 are inserted into the spaces between the teeth of the first roller 17 with play, and the composite sheet 1′ is inserted between the two toothed rollers 17, 17 in this state and thereby subjected to stretchability imparting processing. A configuration may be adopted in which both of the pair of toothed rollers 17, 17 are driven by a driving source (double-rotating rollers), or a configuration may be adopted in which only one of the pair of toothed rollers 17 is driven by a driving source (driving-follower rollers). With regard to the tooth profile of the toothed rollers 17, a commonly used involute tooth profile or cycloid tooth profile can be used. An involute tooth profile or cycloid tooth profile with a reduced face width is preferable. With regard to the elasticity realizing processing, the elasticity realizing processing disclosed in Patent Literature 1 can be used as appropriate.

As a result of the stretchability imparting step, the thickness of the stretch sheet 1 is increased to preferably 1.1 times or greater, and more preferably 1.3 times or greater, preferably 4 times or less, and more preferably 3 times or less, and, specifically, is increased to preferably from 1.1 times to 4 times, and more preferably from 1.3 times to 3 times, with respect to the thickness of the composite sheet 1′ prior to the stretchability imparting step. Thus, the constituent fibers of both of the sheet materials 2 and 3 are plastically deformed and elongated, and consequently the fibers become thin. At the same time, both of the sheet materials 2 and 3 become even bulkier, and therefore have a favorable tactile feel and favorable cushioning properties.

Although the present invention has been described based on a preferred embodiment thereof, the present invention is not limited to this embodiment. For example, the arrangement of the spinning nozzles of the spinning head according to the present invention is not limited to that of the foregoing embodiment, and can be appropriately changed without departing from the gist of the present invention. For example, the number of nozzle rows 12L constituting the staggered arrangement portion 12A is not particularly limited, and may be two as shown in FIG. 5 or may be three or more. The shape of the spinning nozzles 12 in a plan view and the like can be appropriately adjusted depending on the use and the like the stretch sheet, which is an object to be produced, and is not particularly limited.

The stretch sheet for an absorbent article that is produced by performing the production method of the present invention can be suitably used as an outer cover of a pull-on disposable diaper, for example. There is no limitation to this use, and the stretch sheet can also be favorably used as a constituent material of an absorbent article such as a sanitary napkin or a disposable diaper. Examples of the constituent material of an absorbent article include a liquid-permeable sheet (including a topsheet, a sublayer, and the like) that is located closer to the skin side than an absorbent member, a sheet constituting an outer surface of a disposable diaper, and a sheet for imparting elasticity and stretchability to a below-waist portion, a waist portion, a leg-surrounding portion, and the like. The stretch sheet can be used as a sheet that forms a wing of a napkin, and the like. The stretch sheet can be used for a section to which it is desired to impart stretchability, and the like, of sections other than the above-described sections. In the case where the stretch sheet for an absorbent article that is produced by performing the production method of the present invention is to be used as a constituent material of an absorbent article, it is only required to add a step of incorporating the stretch sheet into an absorbent article by joining the stretch sheet to another constituent material (e.g., absorbent member) after the stretchability imparting step.

In this specification, in the case where an upper limit value or a lower limit value, or upper and lower limit values of a numerical value are specified, the values of the upper limit value and the lower limit value themselves are also included. Moreover, even if not explicitly stated, it is construed that all the numerical values, or numerical value ranges, equal to or smaller than the upper limit value of that numerical value or equal to or greater than the lower limit value thereof, or within the range between the upper and lower limit values thereof are described.

As used herein, “a”, “an”, and the like are construed as one or more.

In view of the foregoing disclosure of this specification, it should be understood that various modifications and alterations can be made to the present invention. Accordingly, it should be understood that the present invention can also be implemented in an embodiment that is not clearly stated in this specification, without departing from the technical scope based on the description of the claims.

The entire disclosure of Patent Literature above is incorporated in this specification as a part of the content of this specification.

This application is a national phase application of an international application filed on Apr. 1, 2019 that is based upon and claims the benefit of priority from Japanese Patent Application No. 2018-147827 filed on Aug. 6, 2018, and the entire content of both applications is incorporated in this specification as a part of this specification.

### EXAMPLES

Hereinafter, the present invention will be described in greater detail using examples. However, the present invention is not limited in all respects by the following examples.

### Example 1

A stretch sheet having a similar configuration to that of the stretch sheet shown in FIGS. 1(a), 1(b), and 2 and containing a plurality of elastic filaments including constricted filaments was produced using an apparatus having a similar configuration to the spinning apparatus shown in FIGS. 4 to 6 and an apparatus having a similar configuration to the drawing apparatus shown in FIG. 7. Specifically, a composite sheet was obtained using a spinning apparatus including a spinning head in which all of the spinning nozzles were arranged in a staggered pattern like the spinning head 11, and the composite sheet was made to pass through a drawing apparatus including a pair of toothed rollers in each of which teeth and bottom lands were alternately formed in the circumferential direction, like the pair of toothed rollers 17, as shown in FIG. 7. Thus, stretchability was imparted to the composite sheet, and a stretch sheet whose elastic filaments include filaments having one narrow portion was produced. The spinning nozzle pitch, the resin discharge rate, and the take-up rate of the apparatus that was used were as shown in Table 1 below. The configuration of the filaments having one narrow portion included in the elastic filaments of the produced stretch sheet was as shown in Table 1 below. The following materials and the like that were used:
Elastic filaments: a styrene-based thermoplastic elastomer;
Sheet materials: an air-through nonwoven fabric having a basis weight of 20 g/m² and being composed of a composite fiber (non-elastic fiber with a fiber thickness of 3.3 dtex) in which a core portion is made of PET, and a sheath is made of PE; and
Basis weight of stretch sheet: 56 g/m².

### Examples 2 to 4

A stretch sheet was produced in the same manner with that of Example 1 except that the resin discharge rate and the take-up rate were changed.

### Comparative Example 1

A stretch sheet was produced in the same manner with that of Example 1 except that the resin discharge rate and the take-up rate were changed.

### Comparative Example 2

A stretch sheet was produced in the same manner with that of Example 1 except that a spinning head in which all of the spinning nozzles were linearly arranged in a single row was used.

### Comparative Example 3

A stretch sheet was produced in the same manner with that of Example 1 except that the resin discharge rate, and a spinning head in which all of the spinning nozzles were linearly arranged in a single row with a pitch of 1 mm was used.

### Evaluation Tests:

With respect to the stretch sheets that were produced in Examples 1 to 4 and the stretch sheets that were produced in Comparative Examples 1 to 3, the thickness of each stretch sheet was measured by sandwiching the stretch sheet between flat plates under a load of 0.5 cN/cm² and measuring the distance between the flat plates using a laser thickness gauge manufactured by Keyence Corporation. After each sheet material of the stretch sheet was held in a chuck, the sheet material was peeled apart at a rate of 300 mm/min, and the maximum load at this time was measured using a tensile tester manufactured by Shimadzu Corporation, to thereby measure the peeling strength of the stretch sheet. The stretching and contracting ability of the stretch sheet in a 100% elongation cycle test was measured using the above-described method, to thereby measure the 50% return load and the 50% stretch load of the stretch sheet. The appearance was visually evaluated using evaluation criteria below. Table 1 shows the results.

The appearance of a stretch sheet was evaluated by visually observing the stretch sheet to be evaluated. A stretch sheet in which no hole or the like was observed was evaluated as A, a stretch sheet in which a slight hole or the like was observed was evaluated as B, and a stretch sheet in which a hole or the like was observed was evaluated as C.

With respect to the ratio of the number of constricted filaments to the number of elastic filaments, a cut surface of a stretch sheet that was cut along the width direction was magnified under a microscope manufactured by Keyence Corporation, 100 elastic filaments were observed in the cross-sectional direction, the number of filaments having a narrow portion, of the 100 elastic filaments was counted, and the counted number was calculated as the number of constricted filaments. The number of constricted filaments having one narrow portion, of the above-described constricted filaments was counted, the counted number was used as the number of filaments having one narrow portion, and the ratio of the number of filaments having one narrow portion to the number of constricted filaments was calculated. With respect to the end-to-end distance between adjacent elastic filaments, a cut surface of a stretch sheet that was cut along the width direction was magnified under a microscope manufactured by Keyence Corporation, and the end-to-end distance was measured at 100 random points, and an average value of the measured values was used as the end-to-end distance.

TABLE 1

| | | Ex. 1 | Ex. 2 | Ex. 3 | Ex 4 | Com. Ex. 1 | Com. Ex. 2 | Com. Ex. 3 |
|---|---|---|---|---|---|---|---|---|
| Presence or absence | | Present | Present | Present | Present | Absent | Absent | Absent |
| of constricted | | | | | | | | |
| filament | | | | | | | | |
| Relationship | | Radius < | ← | ← | ← | Radius + | ← | ← |
| between center-to-center | | center-to-center | | | | radius < center- | | |
| distance and | | distance < | | | | to-center distance | | |
| radii of adjacent circles | | radius + radius | | | | | | |
| Ratio of number | % | 21 | 34 | 65 | 52 | 0 | 0 | 0 |
| of constricted filaments | | | | | | | | |
| to number of | | | | | | | | |
| elastic filaments | | | | | | | | |
| Ratio of number | % | 63 | 76 | 94 | 25 | 0 | 0 | 0 |
| of filaments having one | | | | | | | | |
| narrow portion | | | | | | | | |
| to number of | | | | | | | | |
| constricted filaments | | | | | | | | |
| End-to-end distance | mm | 0.48 | 0.56 | 0.70 | 0.80 | 0.37 | 0.84 | 0.92 |
| P (average value) | | | | | | | | |
| between adjacent | | | | | | | | |
| elastic filaments | | | | | | | | |
| Maximum length of | μm | 285 | 271 | 250 | 389 | 0 | 0 | 0 |
| constricted filament | | | | | | | | |
| in width direction | | | | | | | | |
| Nozzle shape | | Staggered | Staggered | Staggered | Staggered | Staggered | Single-row | Single-row |
| Resin discharge rate | m/min | 24 | 18 | 12 | 6 | 32 | 24 | 12 |
| Take-up rate | m/min | 150 | 117 | 75 | 40 | 200 | 150 | 150 |
| Pitch of | mm | 0.5 | 0.5 | 0.5 | 0.5 | 0.5 | 0.5 | 1.0 |
| spinning nozzles | | | | | | | | |
| Thickness of | mm | 0.40 | 0.40 | 0.39 | 0.36 | 0.31 | 0.38 | 0.44 |
| stretch sheet | | | | | | | | |
| Peeling strength | cN/filament | 20 | 18 | 18 | 12 | 22 | 40 | 21 |
| of stretch sheet | | | | | | | | |
| Appearance | | A | A | A | A | B | C | A |
| (hole formation) of | | | | | | | | |
| stretch sheet | | | | | | | | |
| 50% return load | cN/50 mm | 125 | 118 | 124 | 137 | 120 | 116 | 59 |
| of stretch sheet | | | | | | | | |
| 50% stretch load | cN/50 mm | 259 | 258 | 248 | 270 | 271 | 263 | 138 |
| of stretch sheet | | | | | | | | |
| Ratio of 50% | % | 48 | 46 | 50 | 51 | 44 | 44 | 43 |
| return load/50% stretch | | | | | | | | |
| load of stretch sheet | | | | | | | | |

As shown in Table 1, the stretch sheets of Examples 1 to 4 contained constricted filaments, and had a 50% return load equivalent to or superior to that of the stretch sheet of Comparative Example 1, but nevertheless had a larger end-to-end distance (pitch) between adjacent elastic filaments and a larger thickness, compared with the stretch sheet of Comparative Example 1. The stretch sheets of Examples 1 to 4 had a 50% return load equivalent to or superior to that of the stretch sheet of Comparative Example 2, but nevertheless the appearances thereof remained favorable compared with the stretch sheet of Comparative Example 2. Although the stretch sheets of Examples 1 to 4 kept favorable appearances as is the case with the stretch sheet of Comparative Example 3, those stretch sheets had a significantly greater 50% return load.

From these results, it can be seen that, according to the stretch sheet of the present invention, since the constricted filaments are contained therein, it is possible to improve the stress while maintaining a favorable tactile feel and appearance.

From the production conditions that were employed in Examples 1 to 4, it can be seen that a stretch sheet containing constricted filaments and having an excellent tactile feel, appearance, and 50% return load can be efficiently produced by using a spinning head in which spinning nozzles are arranged in a staggered pattern and setting the take-up rate, or the resin discharge rate and the take-up rate, to be within a predetermined rate range.

### INDUSTRIAL APPLICABILITY

According to the stretch sheet of the present invention, it is possible to provide a stretch sheet in which stress can be improved while maintaining a favorable tactile feel and appearance. According to the method for producing a stretch sheet of the present invention, it is possible to provide a method for producing a stretch sheet with which such a stretch sheet can be efficiently produced.

1. A stretch sheet for an absorbent article, in which a plurality of elastic filaments that are arranged so as to extend in one direction without intersecting one another are joined to an extensible sheet material over their entire length in a substantially non-stretched state,
wherein some or all of the plurality of elastic filaments are constricted filaments having at least one narrow portion in a cross section that is orthogonal to an extending direction in which the elastic filaments extend.

2. The stretch sheet for an absorbent article according to claim 1, wherein the cross section of each constricted filament that is orthogonal to the extending direction has a shape in which a plurality of circles are connected together in a partially overlapping state, and
a center-to-center distance between adjacent circles is shorter than the sum of a radius of a first one of the circles and a radius of a second one of the circles, and longer than the shorter radius of the radius of the first circle and the radius of the second circle.

3. The stretch sheet for an absorbent article according to claim 1, wherein the ratio of the number of said constricted filaments to the number of said elastic filaments is from 5% to 90%.

4. The stretch sheet for an absorbent article according to claim 1, which includes a plurality of said constricted filaments, wherein the ratio of the number of constricted filaments having one narrow portion to the number of said constricted filaments is 50% or greater.

5. The stretch sheet for an absorbent article according to claim 1, wherein an average value of an end-to-end distance between the elastic filaments that are adjacent to each other in a width direction is from 0.3 mm to 2 mm.

6. The stretch sheet for an absorbent article according to claim 1, wherein, in the cross section of each constricted filament that is orthogonal to the extending direction, the maximum length of the constricted filament is from 100 μm to 800 μm.

7. The stretch sheet for an absorbent article according to claim 1, wherein the elastic filaments that are joined to the sheet material in the substantially non-stretched state are joined to the extensible sheet material over their entire length in a state in which the elastic filaments do not contract upon removal of external force.

8. The stretch sheet for an absorbent article according to claim 1, wherein a constricted filament having one narrow portion has a pair of depressed portions when viewed in a cross section of the constricted filament that is orthogonal to the extending direction, the depressed portions being depressed from a peripheral surface of the filament toward the inner side of the cross section.

9. The stretch sheet for an absorbent article according to claim 1, wherein a thickness of the stretch sheet for an absorbent article is from 0.32 mm to 0.5 mm.

10. The stretch sheet for an absorbent article according to claim 1, wherein a peeling strength of the stretch sheet for an absorbent article is from 5 cN/filament to 30 cN/filament.

11. The stretch sheet for an absorbent article according to claim 1, wherein the ratio of a load of the stretch sheet for an absorbent article when stretched by 100% along the extending direction of the elastic filaments and returned by 50% from that state to a load of the stretch sheet when stretched by 50% along the extending direction of the elastic filaments is from 45% to 100%.

12. The stretch sheet for an absorbent article according to claim 1, wherein a load of the stretch sheet for an absorbent article when stretched by 100% along the extending direction of the elastic filaments and returned by 50% from that state is from 80 cN/50 mm to 150 cN/50 mm.

13. The stretch sheet for an absorbent article according to claim 1, wherein a load of the stretch sheet for an absorbent article when stretched by 50% along the extending direction of the elastic filaments is from 80 cN/50 mm to 600 cN/50 mm.

14. The stretch sheet for an absorbent article according to claim 1, wherein, with regard to the number of said elastic filaments, a filament that is incorporated in the stretch sheet for an absorbent article while remaining in an immediate post-spinning state, the constricted filament having one narrow portion, and the constricted filament having two narrow portions are each counted as one elastic filament.

15. The stretch sheet for an absorbent article according to claim 1, wherein the narrow portion extends in the extending direction of the elastic filaments.

16. The stretch sheet for an absorbent article according to claim 1, wherein the plurality of elastic filaments are joined to the sheet material through fusion-bonding.

17. An absorbent article comprising the stretch sheet for an absorbent article according to claim 1.

18. A pull-on disposable diaper, wherein the stretch sheet for an absorbent article according to claim 1 is used as an outer cover.

19-38. (canceled)
